(19) United States
(12) Patent Application Publication
(10) Pub. No.: US 2012/0184854 A1
(43) Pub. Date: Jul. 19, 2012

(54) ULTRASONIC VASCULAR FLOW SENSOR WITH TRIANGULAR SENSOR GEOMETRY

(75) Inventors: Balasundara Raju, Chester, NY (US); Eric Cohen-Solal, Ossining, NY (US); Ramon Erkamp, Purdys, NY (US); John Fraser, Woodinville, WA (US)

(73) Assignee: KONINKLIJKE PHILIPS ELECTRONICS N.V., Eindhoven (NL)

(21) Appl. No.: 13/203,065

(22) PCT Filed: Feb. 10, 2010
(86) PCT No.: PCT/IB2010/050613

§ 371 Date: Sep. 26, 2011

Related U.S. Application Data
(60) Provisional application No. 61/154,844, filed on Feb. 24, 2009.

(51) Int. Cl. A61B 8/06

(52) U.S. Cl. 600/454; 600/459

(57) ABSTRACT

An ultrasonic blood flow sensor includes a plurality of adjacent triangular shaped transducer elements which transmit ultrasound waves into a blood vessel and receive reflected ultrasound waves from the blood flow in the vessel. Preferably the transducer elements are paired in pairs of transmit and receive elements. The elements are fixed in a matrix which may be attached in acoustic coupling contact with the skin The matrix retains adjacent transducer elements slightly spaced apart so that the matrix of transducer elements may bend and conform to the shape of the skin surface. The spacing between the triangular elements is neither parallel nor orthogonal to the length dimension of the matrix so that a blood vessel will not be aligned with a space between transducer elements when the matrix is affixed across the location of a blood vessel. In addition the geometry of the elements creates a beam pattern that provide more overlap between transmit and receive beam profiles, thereby increasing the area of sensor coverage.

This is a continuation-in-part of pending U.S. patent application Ser. No. 12/085,133, filed May 15, 2008.

This invention relates generally to the field of ultrasonic blood flow sensors, which find utility in cardiac resuscitation and the guidance of the administration of cardiopulmonary resuscitation (CPR) by measuring vascular blood flow.

In emergencies and during operative procedures, the assessment of the state of blood flow of the patient is essential for both diagnosis of the problem and determining the appropriate therapy for the problem. The presence of a cardiac pulse in a patient is typically detected by palpating the patient's neck and sensing palpable pressure changes due to the change in the patient's carotid artery volume. When the heart's ventricles contract during a heartbeat, a pressure wave is sent throughout the patient's peripheral circulation system. A carotid pulse waveform rises with the ventricular ejection of blood at systole and peaks when the pressure wave from the heart reaches a maximum. The carotid pulse falls off again as the pressure subsides toward the end of the pulse.

The absence of a detectable cardiac pulse in a patient is a strong indicator of cardiac arrest. Cardiac arrest is a life-threatening medical condition in which the patient's heart fails to provide blood flow to support life. During cardiac arrest, the electrical activity of the heart may be disorganized (ventricular fibrillation), too rapid (ventricular tachycardia), absent (asystole), or organized at a normal or slow heart rate without producing blood flow (pulseless electrical activity).

The form of therapy to be provided to a patient without a detectable pulse depends, in part, on an assessment of the patient's cardiac condition. For example, a caregiver may apply a defibrillation shock to a patient experiencing ventricular fibrillation (VF) or ventricular tachycardia (VT) to stop the unsynchronized or rapid electrical activity and allow a perfusing rhythm to return. External defibrillation, in particular, is provided by applying a strong electric shock to the patient's heart through electrodes placed on the surface of the patient's chest. If the patient lacks a detectable pulse and is experiencing asystole or pulseless electrical activity (PEA), defibrillation cannot be applied and the caregiver may perform cardiopulmonary resuscitation (CPR), which causes some blood to flow in the patient.

Before providing therapy such as defibrillation or CPR to a patient, a caregiver must first confirm that the patient is in cardiac arrest. In general, external defibrillation is suitable only for patients that are unconscious, apneic, pulseless, and in VF or VT. Medical guidelines indicate that the presence or absence of a cardiac pulse in a patient should be determined within 10 seconds. For example, the American Heart Association protocol for cardiopulmonary resuscitation (CPR) requires a healthcare professional to assess the patient's pulse within five to ten seconds. Lack of a pulse is an indication for the commencement of external chest compressions. Assessing the pulse, while seemingly simple on a conscious adult, is the most often failed component of a basic life support assessment sequence, which may be attributed to a variety of reasons, such as lack of experience, poor landmarks, or error in either finding or not finding a pulse. Failure to accurately detect the presence or absence of the pulse will lead to adverse treatment of the patient either when providing or not providing CPR or defibrillation therapy to the patient.

Electrocardiogram (ECG) signals are normally used to determine whether or not a defibrillating shock should be applied. However, certain rhythms that a rescuer is likely to encounter cannot be determined solely by the ECG signal, e.g., pulseless electrical activity. Diagnoses of these rhythms require supporting evidence of a lack of perfusion despite the myocardial electrical activity as indicated by the ECG signal.

Thus, in order for a rescuer to quickly determine whether or not to provide therapy to a patient, it is necessary to quickly and easily analyze the patient's pulse, the amount of blood flow, and perhaps the ECG signals in order to correctly determine whether there is any pulsatile flow in the arteries of the patient.

This necessity is particularly dire in situations or systems in which the rescuer is untrained and/or inexperienced person, as is the case with rescuers for which the system described in U.S. Pat. No. 6,575,914 (Rock et al.) is designed. The '914 patent is assigned to the same assignee as the present invention and is hereby incorporated by reference in its entirety. The '914 patent discloses an Automated External Defibrillator (AED) (hereinafter both AEDs and Semi-Automated External Defibrillators—SAEDs—will be referred to jointly as AEDs) which can be used by first-responding caregivers with little or no medical training to determine whether or not to apply defibrillation to an unconscious patient.

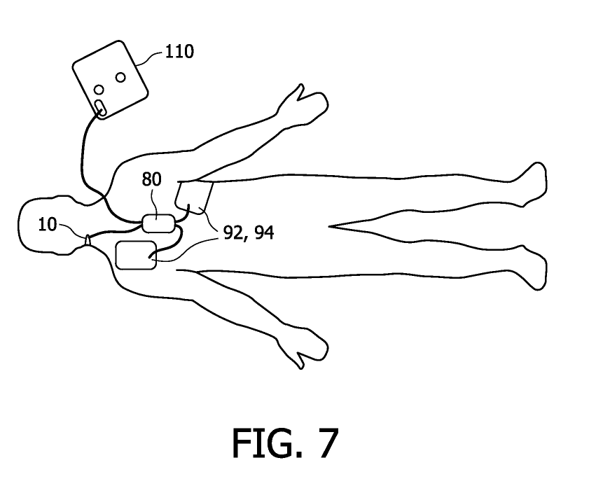

The Rock AED has a defibrillator, a sensor pad for transmitting and receiving Doppler ultrasound signals, two sensor pads for obtaining an ECG signal, and a processor which receives and assesses the Doppler and ECG signals in order to determine whether defibrillation is appropriate for the patient (i.e., whether or not there is a pulse) or whether another form of treatment such as CPR is appropriate. The Doppler pad is secured to a patient's skin above the carotid artery to sense the carotid pulse, which is a key indicator of the sufficiency of pulsatile blood flow. Specifically, the processor in the Rock AED analyzes the Doppler signals to determine whether there is a detectable pulse and analyzes the ECG signals to determine whether there is a “shockable rhythm.” See, e.g., FIG. 7 and accompanying description at col. 6, line 60, to col. 7, line 52, in the '914 patent. The determination of a detectable pulse by the processor in the Rock AED is made by comparing the received Doppler signals against a threshold statistically appropriate with the Doppler signals received. Based on the results of these two separate analyses, the processor determines whether or not to advise defibrillation.

If defibrillation is not advised, the defibrillator can advise that CPR be administered to the patient. When the defibrillator is being operated by a medical professional the medical professional will generally administer CPR in the proper manner. However since an automated defibrillator can be operated by a layperson with no medical training, it is desirable that the defibrillator be capable of coaching the layperson rescuer in the proper application of CPR. CPR coaching can be integrated into a defibrillator as described in U.S. Pat. No. 6,125,299 (Groenke et al.), U.S. Pat. No. 6,351,671 (Myklebust et al.) and U.S. Pat. No. 6,306,107 (Myklebust et al.) The '299 and '671 patents both describe a force sensor which is placed on the patient's chest and to which chest compressions are applied. The force sensor is connected to a defibrillator which senses the applied force of the chest compressions and, using the defibrillator's audible prompts, coaches the rescuer to press “harder” or “softer” or “faster” or “slower.” The '107 patent describes a compression pad with an accelerometer instead of a force sensor which senses the depth of the chest compressions rather than their force. This approach is preferable as CPR guidelines are directed to the depth of compression rather than the applied force, which does not always correlate with compression depth due to different chest resistances to CPR compression.

These techniques are effective for CPR coaching because their quantification capability is directed to measuring the compression of the chest, which causes the lungs to inflate and deflate, thereby at least partially oxygenating the blood. These techniques do not measure the other intended effect of CPR, which is causing at least some circulation of blood. Inducing blood flow to the heart muscle can increase electrical activity in the heart, increasing the probability that a defibrillating shock will restore normal heart rhythm. Inducing blood flow to the brain can lengthen the time before irreversible brain injury is caused by the heart stoppage. Accordingly it is desirable for a CPR measurement system to provide a measure of blood flow to the brain in addition to lung inflation and deflation.

In accordance with the principles of the present invention an ultrasonic transducer pad is provided which is suitable for attachment on the neck above the carotid artery. The transducer pad includes a plurality of transducer elements exhibiting a triangular geometry. The triangular geometry of the elements improves the sensitivity of the transducers to carotid blood flow as it decreases the possibility that the carotid artery will be aligned with a kerf (space) between adjacent transducer elements. In use, a transducer pad of the present invention is attached over the carotid artery and used to sense the flow of blood in the carotid artery during the administration of CPR and/or in conjunction with patient assessment for defibrillation. One or more measures of blood flow are developed from the processing of the ultrasound signals which are used in the guidance of the administration of CPR or cardiac resuscitation.

In the drawings:

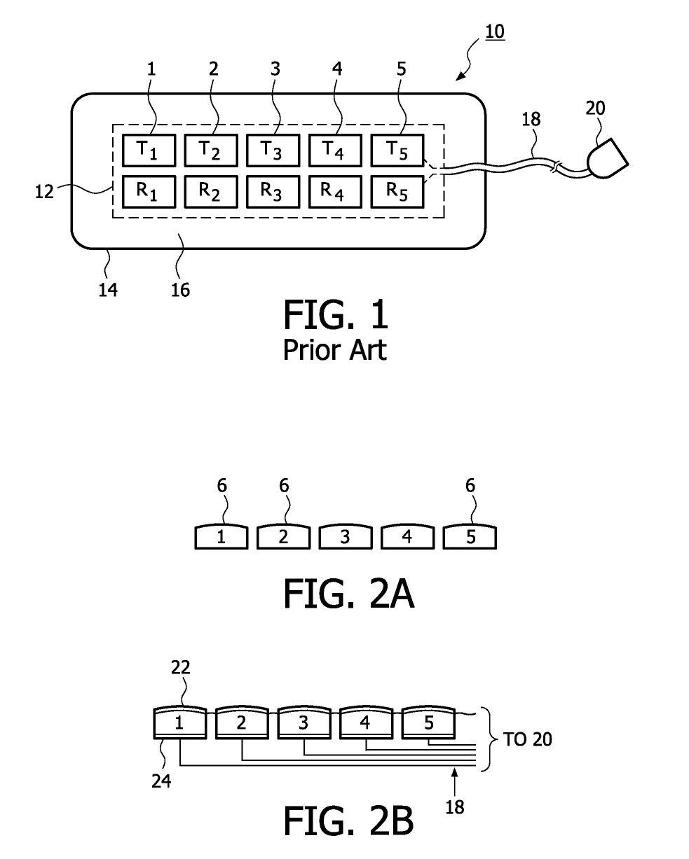

FIG. 1 illustrates a prior art ultrasonic sensor strip for blood flow measurement.

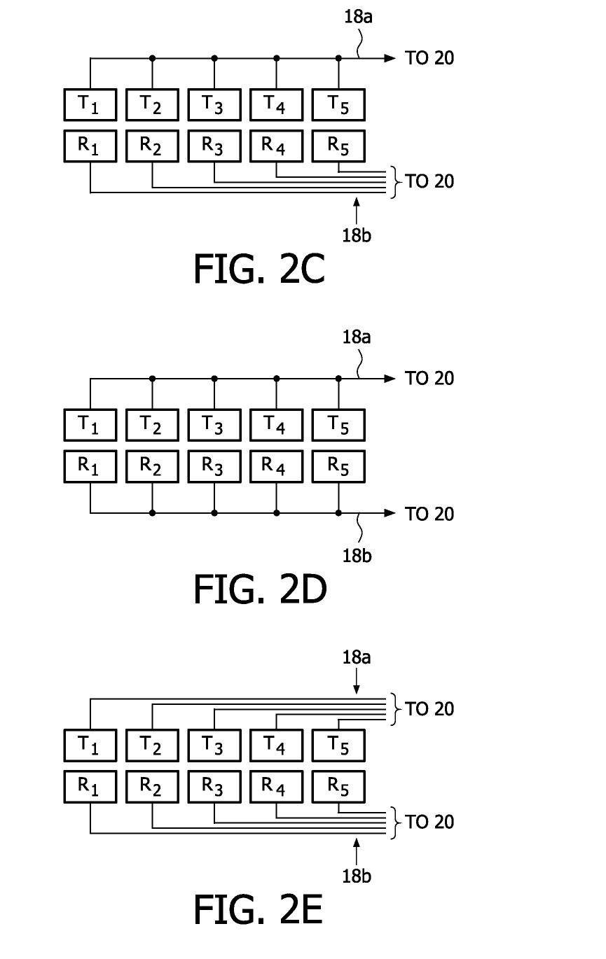

FIGS. 2A-2E illustrate different characteristics and configurations of the transducers of the ultrasonic sensor strip of FIG. 1.

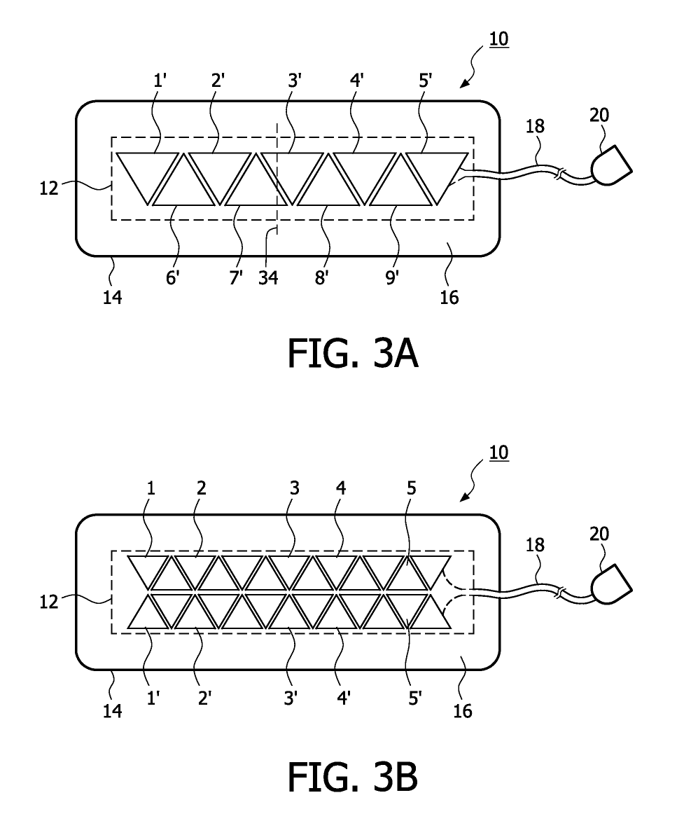

FIGS. 3A and 3B illustrate ultrasonic sensor strips with triangular transducer elements in accordance with the principles of the present invention.

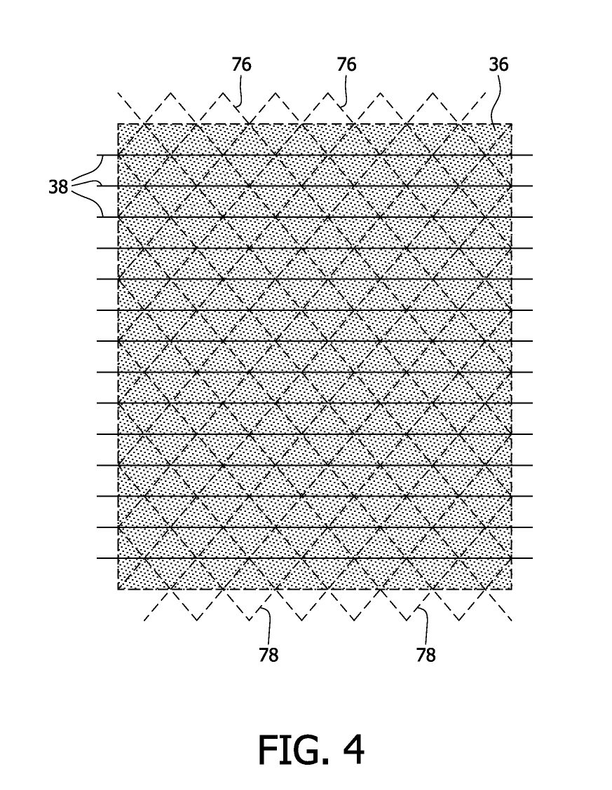

FIG. 4 illustrates a sheet or block of piezoelectric material which is diced to form arrays of triangular sensor elements.

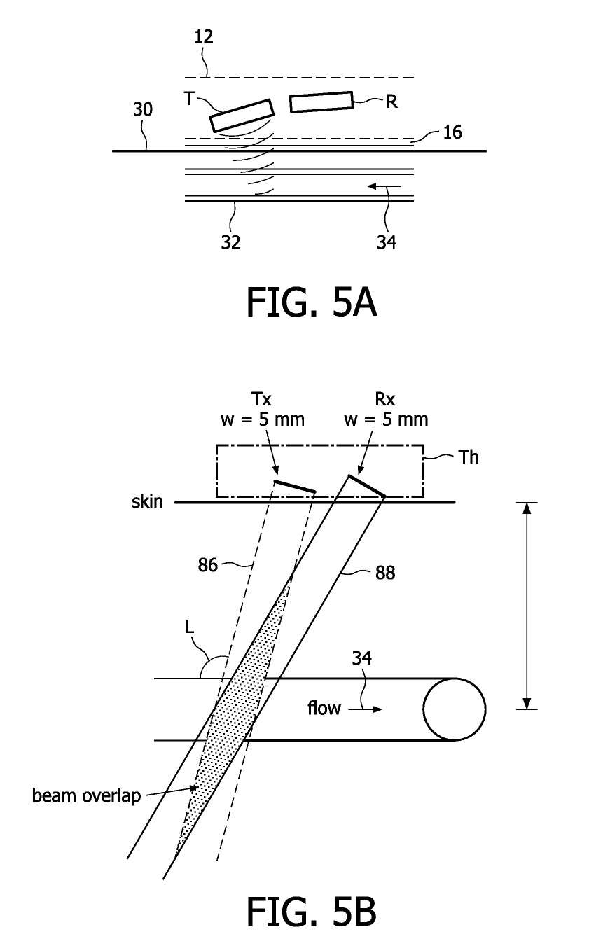

FIGS. 5A-5B illustrate the inclination of the transducers of an ultrasonic sensor strip in accordance with the principles of the present invention.

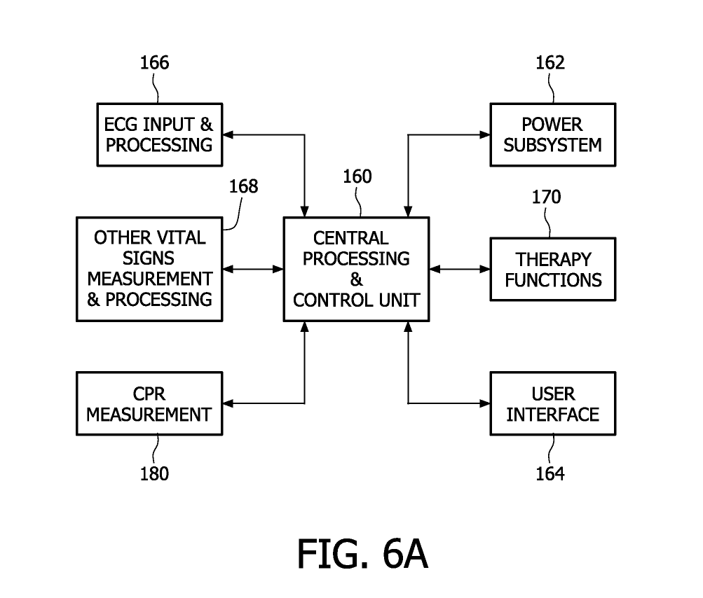

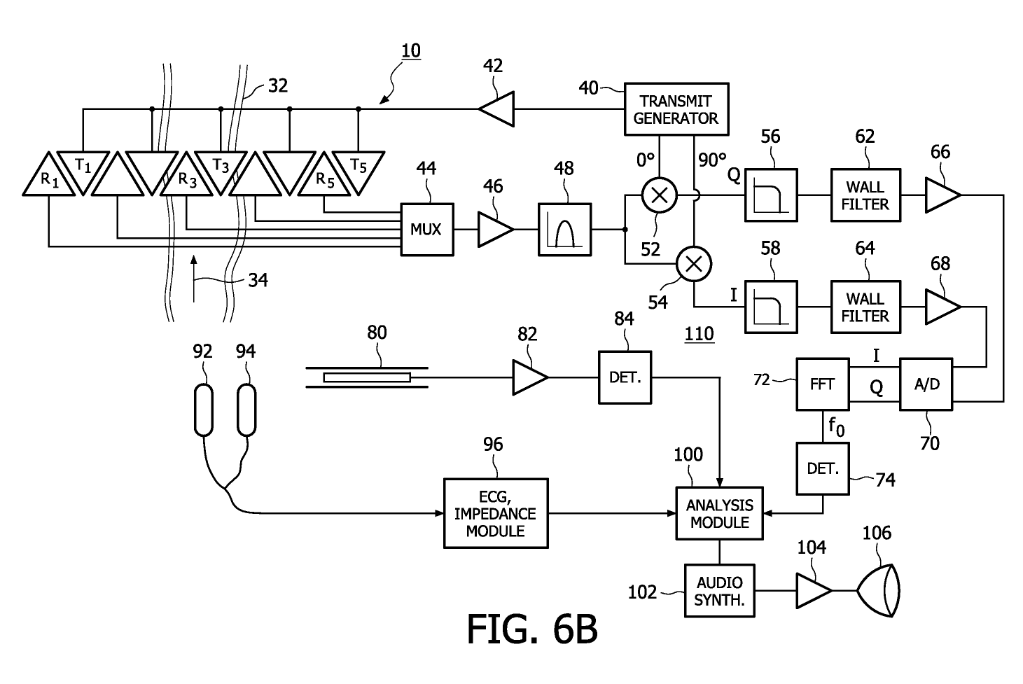

FIG. 6A illustrates in block diagram form a vital signs monitor and therapy system constructed in accordance with the principles of the present invention.

FIG. 6B illustrates in block diagram form a portion of a vital signs monitor and therapy system with pulse detection and CPR guidance constructed in accordance with the principles of the present invention.

FIG. 7 illustrates application of the electrode pads and sensors of the defibrillator system of FIG. 6B during a rescue.

Referring first to FIG. 1, an ultrasonic sensor strip 10 is shown. The sensor strip 10 includes a row of pairs of transducers 1-5. Any number of transducers can be used in a given sensor strip with the number generally being in the range of four to six transducers. Each pair of transducer elements includes a transmitting element (T₁, T₂, etc.) and a receiving element (R₁, R₂, etc.) which enables operation in a continuous wave (CW) ultrasound mode: while the transmitting element is transmitting a wave, the corresponding receiving element is receiving echoes returned in response to the transmission. In this example the transducer elements are unfocussed and individually collimated with a cross-over at a depth of 1.5-2 cm and a range of 0.5-4 cm over which the apertures of the transmit and receive beams overlap so that echoes produced by a transmit transducer element will be received by the corresponding receive transducer element. For pulsed wave (PW) ultrasound operation only a single element is needed which sequentially transmits then receives. The transducers are enclosed in a flexible matrix 12 which can bend to conform to the shape of the skin surface to which the strip is applied. A skin-compatible adhesive such as an electrode gel covers the skin-facing side of the strip and adheres the sensor strip to the skin of a patient. The transducers in the illustrated example are separated by a distance of 1-2 mm so that the row of transducers in the matrix can be bent. The matrix 12 maintains the alignment of the transducers and provides electrical insulation from the body and may be made of silicone (e.g., RTV rubber), for instance. Extending from the matrix 12 is a cable 18 of electrical conductors coupled to the transducer elements as described below. The cable 18 terminates at a connector 20 which connects to a monitoring instrument with which the sensor strip 10 operates. The matrix of transducers is covered by a substrate 14 which adheres the sensor strip to the body. The sensor strip may be attached to the body by an elastic band, a necklace or a Velcro strap. In the illustrated example the substrate is an adhesive tape, or other natural or polymeric material which has an adhesive 16 such as an adhesive electrode gel on its skin-contacting surface. The skin-contacting surface of the matrix of transducers is covered with a material which provides good acoustic coupling between the matrix 12 and the body. This acoustic material may be the same material as adhesive 16 when adhesive 16 has the desired acoustic properties such as the adhesive electrode gel material. The acoustic material may alternately comprise a hydrogel material or an adhesive patch or other solid material.

FIG. 2a is a side view of an example of transducers 1-5. In this example it is seen that the top transmitting surfaces 6 of the transducer elements are rounded. In this example the transducer elements are curved with a 25 mm radius of curvature. The rounding of the transmitting surfaces causes the emitted ultrasound to diverge and thereby insonify a greater area of the body, increasing the likelihood that a target vessel will be insonified and preventing any dead zones between the transducer elements. As an alternative to rounding the shape of the transducer a lens may be used above a flat emitting surface to cause the emitted ultrasound to diverge.

FIG. 2b shows electrical connections made to the transducers 1-5. The transmitting surfaces of the transducer elements which face the skin are covered with an electrode 22 which is grounded for safety. Individual electrodes 22 may be formed on the individual elements which are then electrically connected to the connector 20 by way of cable 18. Alternately the electrode 22 may be a continuous sheet of foil or other flexible, conductive material which covers groups or all of the transducer elements. The sides of the elements which face away from the skin surface have signal electrodes 24 on them. Conductors of cable 18 are connected to these electrodes 24 to provide transmit (drive) signals and return received echo signals from the transducer elements. FIG. 2c is a plan view of the transducer elements showing one example of connection of the signal conductors. In this example all of the transmit elements T₁-T₅ are operated together and electrically connected to one conductor 18a of the cable. The receive elements R₁-R₅ are operated separately and are connected to individual conductors 18b of the cable. This configuration enables all of the transmitting elements to be driven simultaneously by the same transmit wave, with the received echoes being received at the separate receive locations of the receive elements R₁-R₅. FIG. 2d is another example of signal lead connections in which all of the transmit elements T₁-T₅ are driven simultaneously by a transmit signal on conductor 18a, and all of the receive elements R₁-R₅ are electrically coupled together and operated in tandem. All of the echo signals received by all of the receive elements R₁-R₅ at their respective positions are combined and conducted on the same conductor 18b. FIG. 2e is an example of an electrical connection configuration in which each transmit element and each receive element can be operated individually. Each transmit element T₁-T₅ is coupled to its own transmit signal conductor 18a and each receive element R₁-R₅ is coupled to its own receive signal conductor 18b. This example may be preferred when the sensor strip is operated by a battery-powered instrument, as only one transmit element is driven and only one receive channel is needed at any time, thereby conserving battery power.

In accordance with the principles of the present invention, the transducer elements 1′-9′ of the sensor strip 10 have a triangular geometry as shown in FIGS. 3A and 3B. In the prior art embodiment of FIG. 1, which uses conventional rectangular elements, an element pair consists of a transmit element and a receive element and is preferably operated in continuous wave Doppler mode. The sensitivity of the sensor has a significant dependence on exact placement. Generally, to sense blood flow in the carotid artery, the user will apply the sensor strip to the skin of the neck so that the strip is generally orthogonal to the direction of the carotid artery between the chest and the brain. In FIG. 3A this direction of the carotid artery and its blood flow is indicated by the dotted line 34. It has been found that the sensitivity of the transducers is especially reduced when the blood vessel happens to be aligned exactly between pairs of transducer elements. It will be remembered that the transducer pairs are spaced apart so that the sensor strip can flex and bend to accommodate attachment to the curved skin surface of the neck. This loss of sensitivity can be particularly problematic when the user has no a priori information on the location of the blood vessel and happens to place the sensor such that the blood vessel lies in a dead spot between two receive elements. Through the use of triangular shaped receive elements as shown in the example of FIGS. 3A and 3B, the area covered by the element pairs can be made to overlap to a greater degree. In FIG. 3A, transmit and receive elements alternate, whereas in the preferred implementation of FIG. 3B there is a row of dedicated transmit elements 1-5 and so forth, and a row of dedicated receive elements 1′-5′ and so forth. This overlap of coverage reduces the low sensitivity areas between pairs, leading to better sensitivity and higher tolerance to placement inaccuracies. As FIGS. 3A and 3B show, with the triangular geometry the directions of the spaces between the elements are neither parallel nor orthogonal to either the major (length) or minor (width) dimensions of the sensor strip 14, improving the chances that no space will be aligned with the blood vessel when the sensor strip is attached across the presumed direction of the blood vessel. When a blood vessel with an orientation as shown by the dotted line 34 is insonified by an ultrasound wave in the area underneath the strip receive aperture, the reflected ultrasound signal will be picked up with good sensitivity by at least one and in most cases 2 receive elements. This holds for any left-to-right and depth position of the vessel. With rectangular elements there would be a dead-zone when the vessel is in-between two receive elements, but the arrangements of FIGS. 3A and 3B provide a greater certainty of overlap. Moreover, the point of each triangular element will produce a rapidly diverging beam, whereas the base of each triangular element will exhibit a more collimated beam. This beam pattern also ensures reliable insonification regardless of the position of the blood vessel beneath the sensor strip.

A triangular-shaped sensor strip as shown in FIGS. 3A and 3B has some favorable manufacturing aspects. One suitable piezoelectric material for the transducer elements is PZT ceramic, which is available in bars or sheets and may be diced into individual transducer elements with a dicing saw. For dicing of the triangular shaped elements there are only three different saw cut angles along which dicing needs to be performed, as shown in FIG. 4. In this illustration the shaded sheet 36 of PZT ceramic is diced horizontally as indicated by kerf cuts 38 and at two 45° angles as shown by the dashed dicing lines 76 and 78. There is little excess material lost in the process at the edges of the sheet. Another embodiment would be to use tightly spaced trapezoidal-shaped elements. The trapezoidal shape also generates overlap, but a trade off can be made between amount of overlap and the required number of elements needed to cover an area.

FIG. 5a shows one example of how the transducer elements of a transducer pair may be positioned in the matrix 12 for improved signal reception. A Doppler ultrasound signal is angle-dependent. When the angle between the direction of the ultrasound beam and the direction of blood flow is 90°, the Doppler signal is at a minimum, and is strongest when the direction of blood flow is directly toward or away from the transducer. Since vessels close to the skin surface 30 such as the carotid artery 32, which is at an average depth in the body of 20 mm, are approximately parallel to the skin surface, a transducer orientation which transmits ultrasound waves normal to the skin surface 30 will have an angle of incidence of approximately 90°to the direction of flow. To reduce the probability of this orthogonal beam-to-flow-direction orientation the transducer elements are inclined at a shallow angle as shown in FIG. 5a. This relationship between ultrasound beam direction and flow direction is shown in greater detail in FIG. 5b. With the transmitting element Tx inclined as shown, it is seen that an acute angle is formed between the direction 86 of wave travel and the blood flow direction 34 as indicated in FIG. 5b. In FIG. 5b the transducer elements Tx and Rx are angularly offset from each other by an angle of 15°. The transmit beam is at an angle of 75° to the direction of blood flow and the receive beam is at an angle of 60°. This angulation causes the transmit and receive beams 86 and 88 to overlap at the expected depth of the blood vessel as shown by the beam overlap region in FIG. 5b.

In the example of FIGS. 5a and 5b the element inclination angles the beam direction laterally with respect to the length dimension of the row of transducers, effectively causing the transducers to look to the side of the sensor strip. This works well when the sensor strip 10 is positioned across a blood vessel such as across the carotid artery 32 as illustrated in FIG. 6b. Positioning a sensor strip 10 across (orthogonal to) a blood vessel provides a layperson user with the greatest chance of intersecting the unseen vessel with ultrasound. The transmit transducer element aperture is thus looking toward or away from the direction of flow in the carotid artery 32. When the sensor strip is positioned as shown in FIG. 6b the strongest Doppler signal will be detected by the transducer pair T₃-R₃, which is positioned over the carotid artery 32 while the other transducer pairs are not over the vessel. In the Rock system shown in FIG. 4 of the '914 patent the row of transducers is aligned generally parallel to the length of the vessel. An advantage of this placement is that signals will be received by multiple transducer elements, increasing the signal to noise ratio, since multiple transducers are positioned over the blood vessel. A disadvantage is that, if the user misjudges the location of the blood vessel and positions the transducers parallel to but not over the hidden blood vessel, little or no signal will be received. The example sensor strip placement of FIG. 6B will improve the likelihood of success for the layperson user.

FIG. 6A is a block diagram of a vital signs monitor and therapy system constructed in accordance with the principles of the present invention. A central processing and control unit 160 controls the various functions and components of the system and processes vital signs data. The central processing and control unit executes processing and control algorithms appropriate for the vital signs being monitored and the treatment being carried out by the system. The central processing and control unit may be connected to other devices by wired or wireless LAN connections or Bluetooth connectivity. The central processing and control unit 160 and other electronic components of the system are powered by a power subsystem 162 which may include a battery, a.c. line, power supply, and other power management and control functions. The clinician interacts with the system by means of a user interface 164 which may include elements such as a display, audio input and output, keypads, and a printer. The patient's ECG is monitored and processed by an ECG input and processing subsystem 166 which can perform such functions as impedance, ventilation and arrhythmia analysis. The system includes elements for other vital signs measurement and processing 168 such as SPO2, ETCO2, IBP NIBP, and others. The system includes therapy functions 170 such as pacing and defibrillation, high voltage systems, and patient isolation. The performance of CPR is measured by a CPR measurement subsystem 180 as described more fully below.

FIG. 6B illustrates in block diagram form a portion of a vital signs monitor and therapy system which uses a sensor strip 10 of the present invention to help guide the administration of CPR. The sensor strip 10 in FIG. 6B is wired with the transmit elements T₁-T₅ connected in common and the receive elements R₁-R₅ with separate outputs as previously shown in FIG. 2C. Another embodiment would have one row of dedicated receive elements and another row of dedicated transmit elements electrically coupled together as shown in FIG. 3B, which may be preferred in many implementations. The sensor strip 10 is connected to a defibrillator 110, one of the therapy functions 170, which includes the following elements shown in the drawing. A transmit generator 40 generates transmit waveforms for the transmit elements of the sensor strip 10. The transmit waveforms exhibit a nominal frequency in the range of 3-7 MHz and in this example have a nominal frequency of 5 MHz, which is typical for vascular ultrasound applications. The transmit waveforms are amplified by an amplifier 42 and applied to the transmit transducer elements T₁-T₅. The receive transducer elements R₁-R₅ are coupled to a multiplexer 44 which couples the signals received by one of the receive transducer elements to its output. The selected receive signal is amplified by a low noise amplifier 46 and filtered by an r.f. bandpass filter 48. The receive signal is mixed down to baseband by mixers 52 and 54 which are driven in quadrature by reference signals referenced to the transmit waveform. The demodulated quadrature signals are labeled as I and Q in the drawing and comprise quadrature detected components of the Doppler flow vector. The I and Q signals are filtered by lowpass filters 56 and 58 and then filtered by thump filters or wall filters 62 and 64, which pass the flow velocity components to the exclusion of DC (stationary tissue) components and components from the vessel walls. The filtered quadrature components are filtered by Doppler filters 66 and 68 and applied to the two inputs of a dual analog to digital converter 70 which digitizes the Doppler signals. The Doppler signals are translated to the Doppler spectrum by a fast Fourier transform (FFT) processor 72. FFT processing for Doppler signals is well known in the art with different implementations described for instance in “Discrete-Time Signal Processing,” by Oppenheim & Schafer (Prentice Hall, 1989). In a typical implementation consecutive overlapping sequences of Doppler samples are loaded into sliding sample window registers padded with zeroes and processed to produce Doppler frequency signals f_{D} in a Doppler spectrogram centered around zero (DC) and bounded by ±½ the Doppler sampling frequency determined by the transmission interval rate, which is generally in the kiloHertz range. If not done by the FFT processor the amplitude of the Doppler signals is detected by a detector 74 to produce power Doppler output signals.

The power Doppler signals are coupled to an analysis module 100, included in the CPR measurement subsystem 180, which can analyze the Doppler signals in various ways. In one example the multiplexer 44 selects the signal from a different receive transducer element every 10 msec as described in international patent application publication WO 2006/003606, the contents of which is incorporated herein by reference. The multiplexer first selects the signal from nearby transmit elements. After this first sampling period the multiplexer selects the signal from element R₂. The multiplexer continues by selecting signals from elements R₃, R₄, and R₅, then repeats the sequence. During this time the analysis module 100 is looking for a strong power Doppler signal which exceeds a given threshold, such as a predetermined noise level. A valid power Doppler signal is recognized as one which exceeds the threshold by a given signal to noise ratio. In this example the defibrillator system is sampling the power Doppler signals while CPR is performed on the patient. When the rescuer compresses the chest of the patient an amount of blood is forced out of the heart and the pressure wave will emanate through the vascular system, generally causing a pulsatile flow of blood in the carotid artery. The onset of this blood flow is detected during the polling sequence and, when recognized as a valid power Doppler signal by the analysis module, the multiplexer stops polling and continuously couples the valid Doppler signal to the system. In this example the valid Doppler signal is detected by receive transducer element R₃ which is immediately above the carotid artery 32. The signals from receive element R₃ are then continuously sampled by the system. The Doppler frequency f_{D} of the valid signal indicates the flow velocity and the peak signal indicates the maximum instantaneous flow rate caused by the CPR.

The sampling sequence effected by the multiplexer 44 may exhibit any of a number of variations. For instance, if the analysis module senses a decline in the strength of the power Doppler signal from a selected receive element, the multiplexer may be controlled to begin sampling the signals from the receive elements on either side of the selected element to try to find a stronger signal at an adjacent receive element. If a stronger Doppler signal is not found at either of these adjacent transducer locations the multiplexer will return to sampling the signal from transducer element R₃. If multiple processing channels are available in a given device, multiple transducer elements can be monitored simultaneously and the strongest Doppler signal used for analysis.

In addition to detecting velocity, the period of the Doppler waveform is sensed by detecting the recurring peak velocity over several chest compressions. The periodicity of this rate of recurrence indicates the rate of chest compressions during CPR. As a result of this analysis the rescuer is audibly and/or visually coached to administer CPR properly. For instance, a typical CPR protocol may call for the rescuer to administer 15 compressions at the rate of 100 compressions per minute. If the rate of recurrence sensed by the analysis module is less than this desired rate the analysis module will apply a signal to an audio synthesizer 102 or the display screen to issue a verbal “press faster” instruction. The audio synthesizer will produce an audio signal which is amplified by an amplifier 104 and applied to a loudspeaker 106 which audibly instructs the rescuer to “press faster.” The analysis module will also compare the peak velocity of blood flow during the compressions to a desired minimum blood flow velocity to be attained by each chest compression. For instance a typical peak velocity value is about 1 m/sec. The reference used by the analysis module may be less than this nominal rate and if the desired reference velocity is not being attained the analysis module can issue a “press harder” command through the audio synthesizer and loudspeaker of the user interface 164. A visual display such as a row of LEDs or a graphical display can illustrate visually the strength of the flow signal in absolute or relative terms and/or the position along the row of transducer sensors where the strongest flow signal has been detected.

In addition to detecting the peak velocity and period of the Doppler waves the analysis module may produce other measures of the sufficiency of the blood flow caused by the CPR compressions, such as mean velocity, volume flow rate, pulsation index, and flow index as described in international patent application publication WO 2006/030354, the contents of which is hereby incorporated by reference.

The systems of FIGS. 6A and 6B have other sensors which may be used in combination with the Doppler flow sensor to judge the effectiveness of CPR. A compression pad 80 is shown in FIG. 6B which is placed on the patient's chest and against which the CPR compressions are applied. The compression pad includes a force sensor as shown in U.S. Pat. No. 6,351,671 or preferably an accelerometer as described in U.S. Pat. No. 6,306,107. Each time compression is applied to the pad 80 a signal is produced which is amplified by an amplifier 82 and detected by a detector 84. The detected chest compression signal is then used in combination with the information derived from the

Doppler flow signals. For instance, each occurrence of a compression signal should correlate in time with the sensing of a valid Doppler flow signal by the sensor strip 10. Thus, the compression signal can be used to time gate the analysis of the Doppler signal or to correlate and confirm the rate of compression periodicity sensed by the analysis module. The ECG signal, when present, can also be used as a time gate. The amplitude of the force or twice-integrated acceleration signal is a measure of the compressive force or compression depth of the applied compression and can be used in deciding whether to issue a “press harder” or “press softer” command. For instance, while a low flow velocity or volume flow rate may indicate that the rescuer should press harder, the compression signal may show that the rescuer is already pressing as hard or as deep as is safely done on a patient. The analysis module may then withhold the “press harder” command in consideration of this compression information.

The system of FIG. 6B also has chest electrodes 92, 94 which are adhered to the patient's chest and used to sense the patient's ECG signals and thoracic bio-impedance, and to deliver a defibrillating shock. The ECG and impedance signals are processed by an

ECG, impedance module 96 and coupled to the analysis module where they may be used to assist in CPR coaching. For instance, as explained in the '671 patent, the impedance signal will exhibit a change when the chest is compressed and again when the compressive force is relaxed. The times of occurrence of these impedance changes can be used to correlate with or time-gate the Doppler signal analysis to confirm or improve the detection of these signals and the appropriateness of CPR coaching commands.

FIG. 7 illustrates the outline of a patient and shows the defibrillator 110 with the proper placement of the sensor strip 10 on the neck across the carotid artery, the compression pad 80 in the center of the chest, and the electrodes 92,94 placed in the customary positions for ECG measurement and defibrillation. It will be apparent to one skilled in the art that the analysis module can correlate or combine signals from all of these sensors to better produce coaching commands for CPR. It is also possible to combine the sensor strip 10 and the upper defibrillation electrode 92 into one electrode which is placed on the patient's neck as described in US patent publication 2003/0199929.

Other variations of the sensor strip configuration will occur to those skilled in the art. For instance, it may be desirable to use different shaped transducer elements. For instance, the transmit elements could be rectangular and the receive elements triangular, or the reverse.

1. An ultrasonic sensor strip for sensing blood flow comprising:
an ultrasonic transducer assembly of triangular shaped transducer elements;
a connector electrically coupled to the transducer elements;
a flexible matrix which retains the transducer elements in a predetermined spaced arrangement; and
an attachment material which is adapted to attach the matrix in acoustic coupling contact with a subject,
wherein the sensor strip further exhibits a length dimension and a width dimension, wherein the spaces between adjacent ones of the triangular shaped transducer elements exhibit a length which is not parallel to either the width dimension or the length dimension.

2. (canceled)

3. (canceled)

4. The ultrasonic sensor strip of claim 1, wherein the transducer elements have an equilateral triangular shape.

5. The ultrasonic sensor strip of claim 1 wherein the predetermined spaced arrangement further comprises a single row of transducer elements extending in the length dimension of the sensor strip; and
wherein each triangular shaped transducer element in the row has an edge aligned generally parallel to the edge of an adjacent transducer element and spaced apart from the adjacent transducer by 0.5 to 5.0 mm.

6. The ultrasonic sensor strip of claim 1, wherein transducer elements in the transducer assembly are paired with one transducer element being electrically coupled to be a transmitting element and the other element of the pair being electrically coupled to be a receiving element.

7. The ultrasonic sensor strip of claim 6, wherein the paired transducer elements are oriented in the matrix to have overlapping beam patterns at the expected depth of a blood vessel from the skin surface of a subject.

8. The ultrasonic sensor strip of claim 7, wherein the paired transducer elements are further oriented to intersect a blood vessel which is parallel to the skin surface at an angle which is not an orthogonal angle.

9. A Doppler ultrasound system coupled to the connector of the ultrasonic sensor strip of claim 1, the system providing transmit signals to transducer elements of the assembly and receiving reflected ultrasound signals received by the transducer elements from blood in response to the transmit signals.

10. The Doppler ultrasound system of claim 9, wherein the transducer elements are paired in transmit and receive pairs,
wherein the Doppler ultrasound system causes the transmit element of a pair to transmit ultrasound waves and wherein the Doppler ultrasound system operates to receive ultrasound signals from the receive element of the pair.

11. The Doppler ultrasound system of claim 9, wherein the transducer elements are paired in transmit and receive pairs,
wherein the Doppler ultrasound system causes the transmit elements of the pairs to transmit ultrasound waves and wherein the Doppler ultrasound system operates to receive ultrasound signals from the receive elements of the pairs.

12. The Doppler ultrasound system of claim 11, wherein the Doppler ultrasound system is further operable, following reception of ultrasound signals from one of the receive elements, to continuously receive ultrasound signals from that receive element.

13. (canceled)

14. (canceled)

15. (canceled)
